(19) United States
(12) Patent Application Publication
(10) Pub. No.: US 2008/0004497 A1
(43) Pub. Date: Jan. 3, 2008

(54) Substantially non-fluorescent speculums and methods related thereto

(76) Inventors: Peter Whitehead, West Vancouver (CA); Pierre Lane, Vancouver (CA)

Correspondence Address: GRAYBEAL, JACKSON, HALEY LLP, 155 - 108TH AVENUE NE, SUITE 350, BELLEVUE, WA, 98004-5973, US

(21) Appl. No.: 11/402,284

(22) Filed: Apr. 10, 2006

Related U.S. Application Data
(60) Provisional application No. 60/669,848, filed on Apr. 8, 2005.
(60) Provisional application No. 60/785,922, filed on Mar. 24, 2006.

(51) Int. Cl. A61B 1/00

(52) U.S. Cl. 600/184

(57) ABSTRACT

Devices, systems and methods providing speculums constructed to be substantially or completely non-fluorescent. The speculums can be, for example, constructed entirely of non-fluorescent material such as very dark (e.g., black, dark charcoal gray, navy blue) plastic. The speculums can also be any given material and coated with a substantially non-fluorescing material such as a low-fluorescent paint or laminate, or otherwise constructed and configured in any desired manner providing the feature(s) discussed herein.

### CROSS-REFERENCE TO RELATED APPLICATIONS

The present application claims priority from U.S. provisional patent application No. 60/669,848, filed Apr. 12, 2005 and U.S. provisional patent application No. 60/785,922, filed Mar. 24, 2006. This and all other references referenced herein are incorporated herein by reference in their entirety and for all their teachings and disclosures, regardless of where the references may appear in this application.

### BACKGROUND

Speculums are well known. See, e.g., U.S. Pat. Nos. 6,761,687, 6,740,031, 6,719,687, 6,712,761, 6,702,740, 6,669,654, 6,595,917, 6,569,091, 6,436,033, 6,432,049, 6,432,048, 6,416,467, 6,416,466, 6,379,299, 5,873,820. However, traditional speculums can sometimes be problematic for use in detecting faint light responses from target tissue, such as fluorescence, including for example autofluorescence and fluorescence from artificial fluorophores introduced to the given site.

Thus, there has gone unmet a need for improved speculums and related methods of making and using speculums, for example for examinations involving the detection of fluorescence or other weakly-emitting light-types. The present systems and methods, etc., provide speculums, methods and the like that provide enhanced fluorescence detection and/or other advantages.

### SUMMARY

The present systems and methods provides speculums constructed to be substantially or completely non-fluorescent. The speculums can be, for example, constructed entirely of non-fluorescent material such as very dark (e.g., black, dark charcoal gray, navy blue) plastic. The speculums can also be any given material coated with a substantially non-fluorescing material such as a low-fluorescent paint or laminate, or otherwise constructed and configured in any desired manner providing the feature(s) discussed herein.

Typically, the substantially non-fluorescent aspect of the speculums includes at least the portion(s) of the speculum in the field of view (or affecting the field of view, for example due to reflection by target tissue of fluorescence from the piece(s) of the speculum) of the examining person or entity (such as a camera and/or colposcope) such as the interior surfaces of the blade(s) of the speculum, particularly the distal interior surfaces of the blade(s).

Thus, In one aspect, the speculums herein comprise strategically located fluorescent markings, which fluorescence can be due to either or both autofluorescence or provided fluorophores (such as fluorescent paint). The markings can provide any desired information to the examining entity, for example a “ruler” to indicate size, or an outline to indicate the periphery of the blade(s) of the speculum, or a reference shape against which target lesions can be inspected.

In one aspect, the discussion herein relates to speculums wherein substantially all surfaces of the speculum within a field of view during an inspection using the speculum can be substantially non-fluorescent, or even completely non-fluorescent. If desired, all surfaces of the speculum affecting the field of view can be also substantially non-fluorescent. The speculums can be made of molded plastic and the entire speculum can be substantially non-fluorescent, and/or can be coated with a substantially non-fluorescent material. The speculums can comprise at least one, two or more fluorescent markings located within the field of view on a distal portion of the speculum, usually the distal tip of one or more of the blades of the speculum. Spaced apart fluorescent markings can indicated a known, specific distance, and the markings can provide a comparative shape configured to assist an investigator to identify a corresponding abnormality such as cancer, pre-cancer, a given infectious disease or other problem.

The speculums can be made of dark plastic, for example dark blue, brown, green, or red, black, and can be low-reflectivity such as a matte finish. The speculums can exhibit less than about 10%, 5%, 3%, 1% or less the level of fluorescence exhibited by healthy tissue in a vagina when both are subjected to a same fluorescence excitation light. The speculums can be vaginal speculums or can be for any other body orifice such as the anus or nostrils.

In another aspect, the discussion herein relates to methods of using or making the speculums herein.

For example, the methods can comprise inspecting a vagina or other body orifice using a speculum as discussed herein, the methods comprising: a) providing the speculum in the vagina; b) directing fluorescence excitation light through the speculum to a target tissue inside the vagina; and, c) observing fluorescent light emanating from the target tissue substantially without interference from fluorescence emanating from the speculum.

As another example, the methods can comprise constructing the speculum such that substantially all surfaces of the speculum within a field of view during an inspection using the speculum can be substantially or completely non-fluorescent. These and other aspects, features and embodiments are set forth within this application, including the following Detailed Description and attached drawings. In addition, various references are set forth herein, including in the Cross-Reference To Related Applications, that discuss certain systems, apparatus, methods and other information; all such references are incorporated herein by reference in their entirety and for all their teachings and disclosures, regardless of where the references may appear in this application. Unless expressly stated otherwise or clear from the context, all embodiments, aspects, features, etc., herein can be mixed and matched, combined and permuted in any desired manner.

### BRIEF DESCRIPTION OF THE DRAWINGS

FIG. 1 depicts a general side view of a substantially non-fluorescent vaginal speculum in an open state.

FIG. 2 depicts a general top view of a speculum looking down on its handle from above its blades, wherein the speculum comprises desired fluorescent markings.

### DETAILED DESCRIPTION

In a one aspect the present devices, systems and methods provide substantially non-fluorescent speculums. The specula can be made out of a black plastic or other low-fluorescence materials for cost effectiveness and for management of extraneous fluorescence. A speculum made from a black or other dark material typically provides absorption of stray incident illumination and fluorescent light. The speculum's autofluorescence is more likely to be absorbed, or to be inhibited from emission in the first place, and what is not absorbed will be outside of the visible spectrum. Use of a highly fluorescent/reflectant speculum, typically made of a clear or light-colored (e.g., white, beige) material for the speculum could create visible fluorescence in the field of view which could be reflected off of tissue reducing contrast and discrimination of cancer or other-diseased tissues from non-cancerous or other-diseased tissues by increasing the background noise. This could also prove to be a problem for the operator to have a glowing fluorescent object in their field of view (even if, typically, at the edge). The essential properties of the speculums herein are that they are substantially non-fluorescent and/or non-reflectant. Thus, speculums made from such materials that are light colored can still be acceptable provided they are made of suitable materials, and likewise even dark-colored speculums that are still highly fluorescent and/or reflectant are not suitable.

In general, optical glasses produce less autofluorescence in the visible part of the spectrum than do plastics. For example, UV grade fused silica produces very low levels of autofluorescence, BK7 produces moderately low levels of autofluorescence, while glasses like SF1, which contain rare earth elements, exhibit high levels of autofluorescence. Plastics also vary considerably in the amount of autofluorescence they exhibit, for example polycarbonate is less autofluorescent than both polypropylene and polystyrene. The chromophores and fluorophores which are mixed with the plastic pellets during fabrication to give the final plastic a particular color, which can influence the autofluorescence properties of the plastic more than the type of raw plastic. Medical devices such as speculums are often colored “bright white” so they are perceived as being ultra clean and sterile. The addition of chromophores to the plastic can only achieve a “dull white” appearance while the addition of the appropriate fluorophores provides a much brighter white. The addition of the fluorophores to achieve the “bright white” color substantially increases the autofluorescence of the device. Thus, in some embodiments herein, such additional fluorophores are not added, or are removed, from the speculums herein.

In some embodiments the material of the speculum can have a matted appearance, or a roughened surface, to reduce and/or prevent unwanted reflections within the vagina or other body cavity.

In certain embodiments, the speculum has a fluorescent level that is less than 10%, 5%, 3%, or 1% that of the level of healthy tissue in the vagina (or other body cavity) subjected to appropriate excitation light, such as about 442 nm or 470 nm light; other wavelengths or wavelength bands can also be used as desired.

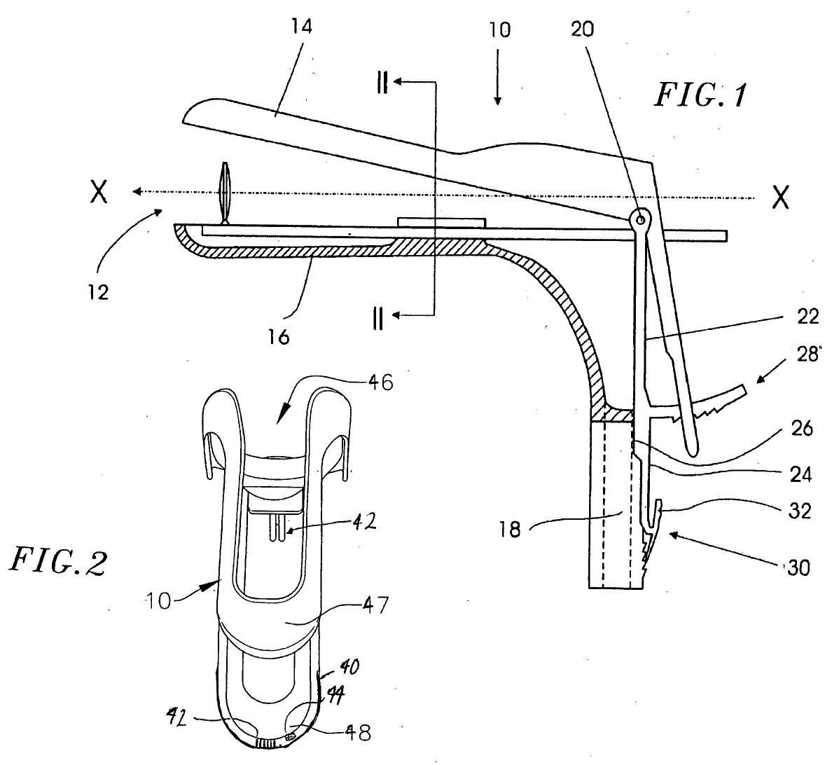

As noted previously, the concepts discussed herein can be used with traditional as well as non-traditional speculums. FIG. 1 is an exemplary traditional speculum from U.S. Pat. No. 6,712,761, and shows a general side view of a vaginal speculum 10 in an open state. In the embodiment shown, the speculum 10 also comprises a single-lens colposcope 12 inserted into the speculum 10. The speculum depicted has been modified such that it is made of a substantially non-fluorescent material such as a substantially non-fluorescent plastic. The speculums herein can be of any desired type such as those that comprise in general two blades insertable into the vagina for spreading and expanding the vagina in a radial direction for observing the conditions of the vaginal walls, cervix and/or other target. The speculums can be suitable for fulfilling treatment procedures, or for conducting appropriate measurements.

In the embodiment shown in FIG. 1, the vaginal speculum comprises a moveable blade 14 pivotally connected to an L-shaped stationary blade 16 having a handle portion 18 arranged substantially perpendicular to the stationary blade 16. Pivotal connection between the blades 14 and 16 is carried out through the intermediary of pivotal axles or pins, only one of which 20 is shown in FIG. 1, since the other one is on the opposite side of the blade not seen in FIG. 1. The pins 20 are rigidly connected to or formed integrally with a fork-like portion 22 on the upper end of a slider 24 slidingly installed in a guide slot (not shown) formed on the rear surface 26 of the handle portion 18.

As a conventional vaginal speculum of the aforementioned type, the speculum 10 of FIG. 1 has on its proximal side a ratchet-type blade rotating mechanism 28 and an upper blade raising mechanism 30 with a ratchet teeth-and-pawl lock device 32.

Fluorescence, such as the autofluorescence of plastic or from fluorophores in fluorescent paint or dye, can also be used to an advantage in the fabrication of measuring devices to be used during fluorescence imaging. For example, a device used to measure the size of cervical lesions (similar to a “ruler on a stick”) can be made from a non-fluorescent plastic with fluorescent plastic over-molded lines to indicate the graduations. If desired, other fluorescent materials/fluorophores can also be used to provide the measurement device. The fluorescence intensity of the graduations is preferably strong enough to provide good contrast but not so strong as to interfere with the fluorescence from the lesion.

FIG. 2 depicts a general top view of a speculum looking down on its handle from above its blades, wherein the speculum comprises desired fluorescent markings. Referring first to the general construction of the speculum in FIG. 2, speculum 10 has a superior cut out 46 That is, as shown in FIG. 2, an upper blade 47 of the speculum is open in its center, which opening extends back toward the proximal end of the upper blade near a handle 42 of the speculum. Thus, the proximal end of the upper blade is cut out to allow a clear field of view during insertion and use of the speculum. Similarly, a lower blade 48 of the speculum is open in its center, which opening extends back toward the proximal end of the lower blade through the transition to the handle. Although, for ease of reference, the blades have been referred to herein as “upper” and “lower,” it should be understood that typically the speculum will be inserted for lateral retraction so that the blades will be at either side of the vaginal wall as inserted for side-to-side retraction.

Lower blade 48 has at its distal end both a fluorescent line 40 indicating the periphery of the blade, and a series of gradated markings 42. In this embodiment, the gradated markings 42 are spaced 2 mm apart; other spacings as desired can be used. The lower blade 48 also has at its distal end a fluorescent comparative marking 44, stylized in the Figure, that provides a comparative shape configured to assist an investigator to identify a corresponding abnormality such as a cancer or precancer.

The shape of vaginal speculum 10 illustrated in FIG. 1 is also shown in U.S. Pat. No. 3,716,047. The shape of speculum 10 illustrated in FIG. 2 is also shown in U.S. Pat. No. 6,702,740. Other exemplary vaginal specula are disclosed in the patents cited above and in U.S. Pat. Nos. 2,579,849; 3,747,591; 3,815,585; 6,048,308.

The present discussion also includes methods of making and using such speculums, including for example in the identification of cancerous or pre-cancerous tissue, or for the identification of suspicious tissue that should be biopsied and further evaluated.

Thus, in one aspect the present discussion provides for making a substantially non-fluorescent speculum wherein the speculum is molded (e.g., injection molded) of substantially non-fluorescent plastic, the method comprising: providing a mold shaped to provide at least one part of a speculum wherein the at least one part is configured to be in or affecting the field of view of an examining entity during examination, providing a formable, substantially non-fluorescent substance such as dark plastic to the mold, and forming the piece(s). The methods can further comprise removing the pieces from the mold and, either at the site of fabrication or at an examination site or elsewhere as desired, combining the pieces to construct the final form of the speculum. In another embodiment, the methods comprise providing at least one part of a speculum wherein the at least one part is configured to be in or affecting the field of view of an examining entity during examination, then applying a layer of substantially non-fluorescent material to the part such that the part, or the relevant portion thereof, is substantially non-fluorescent. In some embodiments, the substantially non-fluorescent material is a paint, a dye or a non-fluorescent plastic laminate.

In another aspect, the methods comprise placing a speculum constructed as discussed herein into an examination area such as a vagina, providing examination light such as fluorescent excitation light to a target area within the examination area, which can be the entire examination area or a portion thereof such as a vaginal wall or cervix, and observing the target area for fluorescence substantially without interfering fluorescence from the speculum. In some embodiments, the methods further comprise comparing the target area with strategically placed fluorescent markings on the speculum. In some embodiments, the examination is carried out by a human being using his or her eyes, or the examination can be carried out using an optical system and a detector, such as colposcope comprising a film or digital camera, or both.

All terms used herein, including those specifically discussed below in this section, are used in accordance with their ordinary meanings unless the context or definition clearly indicates otherwise. Also unless expressly indicated otherwise, the use of “or” includes “and” and vice-versa. Non-limiting terms are not to be construed as limiting unless expressly stated, or the context clearly indicates, otherwise (for example, “including,” “having,” and “comprising” typically indicate “including without limitation”). Singular forms, including in the claims, such as “a,” “an,” and “the” include the plural reference unless expressly stated, or the context clearly indicates, otherwise.

The scope of the present devices, systems and methods, etc., includes both means plus function and step plus function concepts. However, the claims are not to be interpreted as indicating a “means plus function” relationship unless the word “means” is specifically recited in a claim, and are to be interpreted as indicating a “means plus function” relationship where the word “means” is specifically recited in a claim. Similarly, the claims are not to be interpreted as indicating a “step plus function” relationship unless the word “step” is specifically recited in a claim, and are to be interpreted as indicating a “step plus function” relationship where the word “step” is specifically recited in a claim.

From the foregoing, it will be appreciated that, although specific embodiments have been discussed herein for purposes of illustration, various modifications may be made without deviating from the spirit and scope of the discussion herein. Accordingly, the systems and methods, etc., include such modifications as well as all permutations and combinations of the subject matter set forth herein and are not limited except as by the appended claims.

1. A speculum wherein substantially all surfaces of the speculum within a field of view during an inspection using the speculum are substantially non-fluorescent.

2. The speculum of claim 1 wherein all surfaces within the field of view are completely non-fluorescent.

3. The speculum of claim 1 wherein any surface of the speculum affecting the field of view is also substantially non-fluorescent.

4. The speculum of claim 1 wherein the speculum is made of molded plastic and the entire speculum is substantially non-fluorescent.

5. The speculum of claim 1 wherein the speculum is coated with a substantially non-fluorescent material.

6. The speculum of claim 1 wherein the speculum further comprises at least one fluorescent marking located within the field of view on a distal portion of the speculum.

7. The speculum of claim 6 wherein the speculum further comprises at least two spaced apart fluorescent markings, the spacing indicating a known, specific distance.

8. The speculum of claim 6 wherein the at least one fluorescent marking provides a comparative shape configured to assist an investigator to identify a corresponding abnormality.

9. The speculum of claim 8 wherein the abnormality is at least one of a cancer or a pre-cancer.

10. The speculum of any one of claims 1, 2, 5, 6, 7 or 8 wherein the speculum is made of dark plastic.

11. The speculum of any one of claims 1, 2, 5, 6, 7 or 8 wherein the speculum is made of matte black plastic.

12. (canceled)

13. The speculum of any one of claims 1, 2, 5, 6, 7 or 8 wherein the speculum exhibits less than about 5% the level of fluorescence exhibited by healthy tissue in a vagina when both are subjected to a same fluorescence excitation light.

14. (canceled)

15. The speculum of any one of claims 1, 2, 5, 6, 7 or 8 wherein the speculum exhibits less than about 1% the level of fluorescence exhibited by healthy tissue in a vagina when both are subjected to a same fluorescence excitation light.

16. The speculum of any one of claims 1, 2, 5, 6, 7 or 8 wherein the speculum is a vaginal speculum.

17. A method of inspecting a vagina using a speculum comprising:
a) providing the speculum in the vagina;
b) directing fluorescence excitation light through the speculum to a target tissue inside the vagina; and,
c) observing fluorescent light emanating from the target tissue substantially without interference from fluorescence emanating from the speculum.

18. The method of claim 17 wherein the method comprises observing fluorescent light emanating from the target tissue completely without interference from fluorescence emanating from the speculum.

19. The method of claim 17 wherein the method further comprises observing at least one fluorescent marking located on a distal portion of the speculum.

20. The method of claim 19 wherein the method further comprises observing at least two spaced apart fluorescent markings, the spacing indicating a known, specific distance.

21. The method of claim 19 wherein the method further comprises observing at least one fluorescent marking configured to provide a comparative shape, and using the comparative shape to identify a corresponding abnormality.

22. The method of claim 21 wherein the abnormality is at least one of a cancer or a pre-cancer.

23. The method of claim 17 wherein the speculum is made of dark plastic.

24. The method of any one of claims 17, 18, 19, 20, 21 or 22 wherein the speculum is made of matte black plastic.

25. The method of any one of claims 17, 18, 19, 20, 21 or 22 wherein the speculum exhibits less than about 10% the level of fluorescence exhibited by healthy tissue in a vagina when both are subjected to a same fluorescence excitation light.

26. The method of any one of claims 17, 18, 19, 20, 21 or 22 wherein the speculum exhibits less than about 5% the level of fluorescence exhibited by healthy tissue in a vagina when both are subjected to a same fluorescence excitation light.

27. (canceled)

28. The method of any one of claims 17, 18, 19, 20, 21 or 22 wherein the speculum exhibits less than about 1% the level of fluorescence exhibited by healthy tissue in a vagina when both are subjected to a same fluorescence excitation light.

29. A method of making a speculum comprising: constructing the speculum such that substantially all surfaces of the speculum within a field of view during an inspection using the speculum are substantially non-fluorescent.

30. The method of claim 29 wherein the speculum is constructed such that all surfaces of the speculum within the field of view are completely non-fluorescent.

31. The method of claim 29 wherein any surface of the speculum affecting the field of view is also substantially non-fluorescent.

32. The method of claim 29 wherein the method comprises molding the speculum of plastic and the entire speculum is substantially non-fluorescent.

33. The method of claim 29 wherein the method comprises coating the speculum with a substantially non-fluorescent material.

34. The method of claim 29 wherein the method further comprises providing the speculum with at least one fluorescent marking located within the field of view on a distal portion of the speculum.

35. The method of claim 34 wherein the method further comprises providing the speculum with at least spaced apart fluorescent markings, the spacing indicating a known, specific distance.

36. The method of claim 34 wherein the at least one fluorescent marking provides a comparative shape configured to assist an investigator to identify a corresponding abnormality.

37. The method of claim 36 wherein the abnormality is at least one of a cancer or a pre-cancer.

38. The method of any one of claims 30 to 37 wherein method comprises making the speculum of dark plastic.

39. The method of any one of claims 30 to 37 wherein method comprises making the speculum of matte black plastic.

40. (canceled)

41. The method of any one of claims 29, 30, 31, 34, 35 or 36 wherein the speculum exhibits less than about 5% the level of fluorescence exhibited by healthy tissue in a vagina when both are subjected to a same fluorescence excitation light.

42. (canceled)

43. The method of any one of claims 29, 30, 31, 34, 35 or 36 wherein the speculum exhibits less than about 1% the level of fluorescence exhibited by healthy tissue in a vagina when both are subjected to a same fluorescence excitation light.

44. The method of any one of claims 29, 30, 31, 34, 35 or 36 wherein the speculum is a vaginal speculum.
